(19) United States
(12) Patent Application Publication
(10) Pub. No.: US 2023/0142551 A1
(43) Pub. Date: May 11, 2023

(54) LIGHT-CURING COMPOSITION FOR THE PRODUCTION OF DENTAL COMPONENTS WITH MATT SURFACES

(71) Applicant: KULZER GMBH, Hanau (DE)

(72) Inventors: Klaus RUPPERT, Maintal (DE); Alfred HOHMANN, Schmitten (DE)

(21) Appl. No.: 17/916,126

(22) PCT Filed: Mar. 26, 2021
(86) PCT No.: PCT/EP2021/057941

§ 371 Date: Sep. 30, 2022

(30) Foreign Application Priority Data
Apr. 2, 2020 (DE) 10 2020 109 280.6

(51) Int. Cl. C09D 4/06; C09D 135/02; C09D 7/63; A61K 6/17; A61K 6/889; B33Y 70/00; B33Y 80/00

(52) U.S. Cl. CPC C09D 4/06; C09D 135/02; C09D 7/63; A61K 6/17; A61K 6/889; B33Y 70/00; B33Y 80/00; B33Y 10/00

(57) ABSTRACT

The invention relates to a light-curing composition for producing dental components in a DLP method or SLA method, comprising, in relation to the total mass of the light-curing composition: a liquid monomer composition with a mass fraction of 60% or more, comprising one or more radically polymerizable monomers, one or more photoinitiators with a combined mass fraction in the range from 0.001 to 10%, and solid polymer particles with a particle diameter in the range from 0.4 to 4 μm and a combined mass fraction in the range from 0.1 to 30%, wherein the solid polymer particles are dispersed in the liquid monomer composition.

The invention relates to a light-curing composition for the production of dental components in a DLP method or SLA method, a method for producing light-curing compositions, the use of corresponding light-curing compositions for producing dental components with matte surfaces in a DLP method or SLA method and also a kit for producing a corresponding light-curing composition. Furthermore, the use of solid polymer agglomerates in the production of light-curing compositions, the use of solid polymer particles in light-curing compositions for reducing the gloss of dental components obtained by light curing, an uncoated dental component with matte surfaces and a system for producing a dental component in a DLP method or SLA method are disclosed. The subject-matter of the invention is defined in the claims.

Technological progress and advancing digitalization, which have transformed the industrial landscape over the past decade, are also influencing the field of dental technology, fundamentally changing the day-to-day work of dentists and dental technicians. For decades, many types of dental prostheses, such as, for example, dental crowns, bridges, partial and full dentures or inlays, as well as orthodontic treatment devices, for example splints, were primarily produced manually by dental technicians. In particular, the techniques of teeth and jaw molding as well as the production of plaster models were central working steps as a basis for the subsequent dental work.

The traditional process is nowadays enhanced or replaced by the use of computer-aided production and manufacturing methods. This technology is sometimes also referred to by the person skilled in the art as digital prosthetics.

In most cases, so-called CAD/CAM methods are an integral part of computer-aided production. The expression CAD refers to computer-aided design, which refers more broadly to the generation of a digital component that can optionally be directly modified on the computer by the user. The term CAM refers to computer-aided manufacturing, i.e. the conversion of the CAD-generated component into a code which can be used to control a machine, for example a chip-removing machine or a 3D printer. In the field of dental technology, the use of so-called intraoral scanners, which can transmit a precise image of the patient's oral cavity to the computer without contact, has become increasingly established for the creation of the CAD component.

Additive manufacturing methods in which products are manufactured from shapeless materials without the use of special tools and based on the computer data sets generated by CAD/CAM, are particularly important for dental technology. These methods, some of which are also referred to as “rapid prototyping” methods, replace or supplement many working steps in the production of dental components in the field of dental technology today.

Within additive manufacturing methods, various methods have become established in recent years, which differ not only in terms of the apparatus design, but also in particular in the base material used. In many of these so-called 3D printers, solid starting materials, for example in the form of granules or filament, can be processed. What many of these methods have in common is that the component to be produced is built up in layers from layers arranged above one another in order to produce a three-dimensional component.

So-called stereolithography (SLA) and digital light processing (DLP) play a prominent role in additive manufacturing methods. In these methods, a light-curing composition is provided and cured layer by layer at the desired locations by spatially resolved, targeted irradiation. Here, the resulting component is lowered into or lifted out of the composition step by step, for example, such that after each increment there is only a thin film of the light-curing composition over the last layer formed, which film corresponds approximately to the thickness of the next layer to be polymerized. SLA and DLP methods are similar in terms of their basic principle, but are quite different in terms of the apparatus used. In the SLA method, for example, a laser is used which runs successively over the structure to be produced, whereas in the DLP method a suitable projection technique is used to expose an entire surface simultaneously.

The principle of the SLA and DLP methods is known from the prior art and disclosed, for example, in U.S. Pat. No. 4,575,330 A. The use of additive manufacturing methods in dental technology is known, for example, from documents DE 102016107935 A1 and DE 1020122011371 A1 and is also described in EP 3020361 B1. With these methods, it is now possible to process on the computer the component information recorded directly at the patient and to convert this information immediately into a dental component by means of additive manufacturing, which means, for example, that molding and plaster casting can be eliminated in the production of dental components. In addition, even entire partial or full dentures can be produced using SLA and/or DLP methods.

Despite the undeniable usefulness of these methods, which is also reflected in how rapidly this technology has spread, there has long been a substantial problem in the corresponding production of dental components, which has not been satisfactorily solved to date. The components produced in a SLA or DLP method regularly have an excessively high gloss on the surface after curing, i.e. they have the property of reflecting light in a completely or partially specular manner.

While this property is less problematic in many other technological fields or even desirable in some cases, this surface property regularly proves to be disadvantageous in dental technology. The person skilled in the art of dental technology understands that for the intended use of the dental components, a matte surface or at most a surface with a silky matte gloss would be desirable. This is due to the fact that historically a high proportion of the established processes in dental component processing have been adapted to the plaster commonly used for decades and its surface properties, which is also particularly evident in the lower user acceptance of glossy components. Accordingly, a similarity to the conventional plaster model in gloss, reflection behavior and degree of matting of the dental component would be particularly desirable.

The most important aspect in practice is the digital scannability of the component, which can be significantly limited in the case of glossy surfaces. In practice, dental components produced by additive manufacturing methods are machined manually at a later stage, for example, and then have to be re-digitized by suitable scanners. This re-digitization is made significantly more difficult if the surface of the dental component, unlike that of the plaster model, is glossy, meaning that the CAD data obtained are sometimes of unsatisfactory quality or errors occur which make it necessary to carry out the scanning process several times, thus increasing the workload and costs.

The problem that glossy structures are often inadequately scannable is well known in the art. In this respect, the inventors have found that the undesirable tendency towards glossy surfaces is particularly pronounced when certain monomers are used in the light-curing composition. Disadvantageously, the corresponding, usually high-boiling, monomers in the light-curing composition are mostly essential in order to give the dental components obtained therefrom the desired product properties such as mechanical stability or temperature resistance, meaning that they cannot be easily substituted by monomers with a lower gloss tendency.

In the prior art, various solutions have become established for dealing with the problem of glossy surfaces and inadequate scannability. DE 100038564 A1 teaches, for example, the use of a metal powder or a powder with a metallic effect in a molding composition, for example plaster, in order to improve its scannability. In contrast, DE 102006056451 A1 discloses that a molding material should be subsequently roughened in order to improve its scannability. These two solutions cannot easily be transferred to an additive manufacturing method and are therefore regularly perceived as disadvantageous. In particular, the subsequent mechanical roughening of the component is not an option in many cases, even aside from cost aspects, if there is a risk of this changing the exact dimensions of the component or adversely affecting the surface adhesion.

The solutions known from other branches of industry, for example the paint industry, which mostly rely on the addition of a matting agent to a paint, are regularly unsuitable for applications in SLA and DLP methods, since these matting agents are mostly fillers which, after the paint film has dried and cured, are intended to produce an irregular surface and thus lead to diffuse reflection. A prerequisite for this effect is not least the high degree of shrinkage of corresponding paint compositions, which after drying leave behind a surface that is contoured by the fillers. In the case of the monomers used in dental technology, however, a correspondingly high degree of shrinkage during curing is generally not possible, as this would also not be desirable in regard to fine-tuning the component contours. Furthermore, in particular if the precise design of the component contours is important, a significantly roughened surface is often also not desirable for achieving a matting effect, since the increased adhesion to roughened surfaces can make working with the dental component more difficult because, for example, molding or modeling materials adhere excessively.

As an alternative to these approaches, matting liquids have become established in the dental field, as disclosed for example in DE 102013110549 A1. Besides adhesives, these matting liquids also contain pigments, for example titanium dioxide. However, the use of corresponding matting liquids on dental components produced using SLA or DLP methods is regularly perceived as disadvantageous. This is due in particular to the fact that the application of a matting liquid is an additional working step which appreciably extends production time and thus increases costs. Furthermore, in any subsequent 3D scanning step the accuracy of the digitization of the surface geometry of the dental component can be negatively influenced by inhomogeneous layer thicknesses of the matting liquid. Since the dental components to be coated are also regularly medical products, the corresponding use of a matting liquid disadvantageously also requires that the latter also has to be correspondingly approved as a further medical product, which considerably increases the regulatory expense involved in marketing.

US 20140131908 A1 and US 20190053883 A1 disclose compositions which may optionally be designed as light-curing compositions and in which so-called “silicone-acrylate-based rubber impact modifiers” are used, which are designed as core-shell particles. These core-shell particles preferably have a diameter in the range from 0.01 to 100 μm and serve to improve the mechanical properties of the cured compositions.

WO 2018/167213 discloses dental molds which have an opacity of 70 to 78% and contain organic filler and/or a powdery composite material. According to the disclosed exemplary embodiments, the composition for the production of these molds comprises about 66% by weight of polymer particles with average particle sizes in the range from 15 to 60 μm.

Until now, light-curing compositions for use in SLA or DLP methods have not been known which are specifically modified in such a way that the dental components produced therefrom have particularly favorable gloss properties directly, such that they exhibit at most a silky matte gloss on their surface.

The primary object of the invention was therefore to specify a light-curing composition for the production of dental components in a DLP method or SLA method, which makes it possible to produce dental components which, in particular without further treatment, exhibit advantageous gloss properties on their surface, and which thus eliminate or reduce the aforementioned disadvantages of the solutions known from the prior art. The light-curing composition should preferably have a comparatively low, preferably easily adjustable, viscosity in order to enable easy handling in SLA or DLP methods. In addition, the light-curing composition should be curable using typical photoinitiators. Here it was desirable that, in order to achieve the effect, no inorganic additives or fillers are required, in particular no metals or metal oxides, since these can potentially disadvantageously increase the weight and/or the surface hardness and/or the mechanical properties of the components produced, and thus, for example, reduce their machinability. It was very particularly desirable that the dental components produced from the light-curing composition do not have any increased roughness on the surface, which could artificially modify the surface structure desired according to the CAD data and/or lead to an unintentionally strong adhesion of applied molding compositions.

It was an objective of the present invention to enable the light-curing composition to be produced to a large extent from substances which are already regularly used in dental technology. It should also be possible here for the light-curing composition to comprise monomers that have a particularly high tendency to form glossy surfaces. Particularly because of the inconclusive evaluation of the health implications of small nanoparticles, i.e. of nanoparticles with a diameter of 300 nm or less, it was also particularly desirable from a health and regulatory point of view to specify a light-curing composition which does not contain small nanoparticles with a diameter of 300 nm or less.

A secondary object of the invention was to specify a method for producing light-curing compositions by which, in a cost-efficient and reproducible manner, light-curing compositions can be produced from which dental components can be produced by SLA or DLP methods, whose surface exhibits at most a silky matte gloss. Here, the requirement was that the method can be carried out using common devices and that, in particular, only those starting products are used which can be easily handled and stored, wherein, in particular, it was desirable for the operating personnel not to come into contact with small nanoparticles in dry or dusty form when carrying out the method, in order to ensure the highest possible operational safety and also to avoid contaminating the environment.

It was a further object of the invention to specify uses for corresponding light-curing compositions as well as for solid polymer agglomerates and for solid polymer particles.

It can be regarded as a supplementary object of the invention to specify a kit for producing corresponding light-curing compositions, a system for producing a dental component and an uncoated dental component with a matte surface.

The inventors have now recognized that the objects described above can be achieved when solid polymer particles with a particle diameter in the range from 0.4 to 4 μm and a combined mass fraction in the range from 0.1 to 30% are added to a light-curing composition with a high proportion of liquid monomers, if these solid polymer particles are dispersed in the liquid monomer composition.

In the inventors' experiments, it has been reliably shown that by using corresponding light-curing compositions in a SLA or DLP method, dental components are obtained that have a greatly reduced gloss on the surface, leading to the conclusion that there is a basic correlation. Surprisingly, it emerged that the dental components obtained do not display any significantly increased values with regard to surface roughness as compared to dental components to whose light-curing composition no solid polymer particles had previously been added. This indicates that the effect found by the inventors probably does not result from a particularly irregular surface, as is known for example in lacquers. Without wishing to be tied to this theory, it appears that the solid polymer particles are present in the cured material as small, separate domains which are distributed in the material and in which the resulting component has a different chemical composition and, consequently, a different optical refractive or scattering behavior than in the polymerized monomer composition. The inventors hypothesize that it is probably the distribution of these small regions over the surface of the dental component that contributes to the significant matting effect observed experimentally.

Furthermore, the inventors have developed an efficient process by which corresponding light-curing compositions can be obtained. An essential aspect of this method is based on the fact that the solid polymer particles of the liquid monomer composition are not added in isolation, but rather in the form of larger polymer agglomerates, which are sometimes also referred to as agglomerated polymer beads. These polymer agglomerates are added to the liquid monomer composition in order to produce a basic mixture. The solid polymer particles are first produced by mechanically treating the basic mixture in order to break down the solid polymer agglomerates and disperse the polymer particles of the liquid monomer composition, in particular using a three-roll process. This results in a light-curing composition which during further processing can produce the advantageous gloss properties described above.

The objects described above are accordingly achieved by light-curing compositions, methods, uses, systems, kits and uncoated dental components as disclosed below.

Preferred designs according to the invention are apparent from the dependent claims and the following embodiments. Such features, which are referred to below as preferred, are combined with other features referred to as preferred in particularly preferred embodiments. Combinations of two or more of the embodiments referred to below as particularly preferred are thus very particularly preferred. Features referred to below as preferred for light-curing compositions according to the invention are likewise preferred features of corresponding methods and uses.

The invention relates to a light-curing composition for the production of dental components in a DLP method or SLA method, comprising on the basis of the total mass of the light-curing composition:

a liquid monomer composition with a mass fraction of 60% or more, comprising one or more radically polymerizable monomers, preferably consisting of one or more radically polymerizable monomers,
one or more photoinitiators with a combined mass fraction in the range from 0.001 to 10%, and
solid polymer particles with a particle diameter in the range from 0.4 to 4 μm and a combined mass fraction in the range from 0.1 to 30%, wherein the solid polymer particles are dispersed in the liquid monomer composition.

The term “light-curing”, which is used above to characterise the composition, corresponds to the technical term commonly used in the industry. The term is to be understood as the property whereby the composition is cured by the application of electromagnetic radiation, in particular light, in that the radiation induces the polymerization of radically polymerizable monomers in the composition by means of a photoinitiator. The wavelength of the radiation used for this purpose does not necessarily have to lie in the visible light range, but also comprises the adjacent wavelength ranges in the infrared or UV range, wherein wavelengths between 200 and 500 nm, i.e. in the blue and ultraviolet range, are used particularly frequently.

For the purposes of the present invention, the term “dental components” refers to all components and three-dimensional structures which are made in the field of dental technology as an intermediate stage or end product, i.e. irrespective of the underlying dental indication, for example dental models, gingival masks, bite splints, CAD-to-cast molds, impression trays or drilling templates.

The terms “DLP method” and “SLA method” respectively refer to the methods of digital light processing and stereolithography described above, which are known as methods to the person skilled in the art. The specification that the light-curing composition should be suitable for the production of dental components in these methods imposes, in particular, a functional requirement on the viscosity of the material, insofar as this should be sufficiently low. This excludes many light-curing compositions used elsewhere in the dental field, in particular those with high filler contents, for example ceramic slips. The person skilled in the art will readily recognize whether a monomer composition is qualitatively liquid. In the context of the present invention it is assumed that any monomer composition having a dynamic viscosity of 10 Pa s or less, preferably 5 Pa s or less at 23° C., can be referred to as liquid.

The mass fractions defined above for the liquid monomer composition, the photoinitiators and the solid polymer particles are in each case based on the total mass of the light-curing composition. The person skilled in the art understands that the mass fractions are defined with the proviso that the total mass fractions of the light-curing composition add up to 100%. This means that if, for example, an additive with a mass fraction of 10% is added to the composition, the photoinitiators and the solid polymer particles may together have a mass fraction of only 30% or less.

The radically polymerizable monomers are characterized by their property of crosslinking with each other in a chain reaction upon contact with a free-radical initiator, wherein the free-radical initiator in light-curing compositions is regularly provided by the photoinitiator(s). In terms of the basic principle, the present invention is not limited to certain radically polymerizable monomers, but can be applied to all radically polymerizable monomers, most of which have a terminal unsaturated double bond via which the radical polymerization can proceed. In the field of dental chemistry, however, (meth)acrylates are of paramount importance as monomers; the term (meth)acrylates, as understood by the person skilled in the art, denotes both acrylates and methacrylates. These monomers, which are frequently used in dental chemistry, are known to the person skilled in the art and are disclosed, for example, in EP 3020361 B1 or DE 3941629 C1. These monomers, which may be monofunctional or multifunctional, are selected by the person skilled in the art on the basis of the physicochemical and application properties desired for the dental component to be produced.

Photoinitiators are selected by the person skilled in the art on the basis of the radically polymerizable monomers used and the desired wavelength range for the polymerization, and said person can easily refer to information tables and/or information provided by the manufacturer. In the context of the present invention, the term “photoinitiator” also includes auxiliary initiators, co-initiators or accelerators. Suitable photoinitiators are disclosed, for example, in EP 3020361 B1 or WO 95/13565 A1.

The solid polymer particles to be used according to the invention in the light-curing composition have a particle diameter in the range from 0.4-4 μm. It has been shown experimentally that even small amounts of this additive lead to a considerable improvement in the gloss properties of the dental component to be produced from the light-curing composition. According to the invention, the polymer particles are solid, i.e. already substantially polymerized, i.e. at least 80%, preferably at least 90%, particularly preferably at least 98% polymerized.

According to the invention, the solid polymer particles are dispersed in the liquid monomer composition. This means that there is substantially no concentration gradient of solid polymer particles in the light-curable composition between two macroscopic volume portions. This preferably means that the differences in concentration between two separate macroscopic volumes of the composition of one milliliter in the light-curing composition in each case show less than 10%, preferably less than 5%, particularly preferably less than 2%, of deviation in the concentration of solid polymer particles. For the purposes of the present invention, the solid polymer particles are in particular not dispersed in the liquid monomer composition if they are floating on the surface of the liquid monomer composition, as has been observed, for example, for agglomerated particles in polymer agglomerates for which the advantageous effect of the invention has not been shown.

Accordingly, light-curing compositions are preferred in which the solid polymer particles are substantially not agglomerated. This means that preferably less than 50%, particularly preferably less than 20%, very particularly preferably less than 10%, of the polymer particles are present in the agglomerated state. However, the person skilled in the art will understand that in the case of correspondingly small polymer particles, local formation of small agglomerates can always occur, meaning that they do not have to be completely ruled out, wherein these agglomerates preferably comprise a maximum number of 10 solid polymer particles.

Preference is given to a light-curing composition according to the invention, wherein the light-curing composition does not contain metallic particles or metal oxides.

The light-curing composition according to the invention makes it possible to produce dental components in an SLA or DLP method which, without further treatment, exhibit advantageous gloss properties on their surface, i.e. a low gloss. In this respect, the light-curing composition according to the invention has a comparatively low viscosity due to the high proportion of liquid monomer composition and, at the same time, easily adjustable viscosity due to the polymer particles. At the same time, the light-curing composition according to the invention is curable using typical photoinitiators and can be produced to a large extent from substances that are already regularly used in dental chemistry, even from monomers with a high gloss tendency. Advantageously, no inorganic additives or small nanoparticles with a diameter of 300 nm or less are required to achieve the effect, and the dental components produced from the light-curing composition do not have significantly increased roughness on the surface.

Preference is given to a light-curing composition according to the invention, wherein the solid polymer particles consist of polymers of monomers selected from the group consisting of monofunctional (meth)acrylates and polyfunctional (meth)acrylates, preferably consisting of polymethyl methacrylate, wherein the solid polymer particles preferably consist of polymers of monomers that are not part of the liquid monomer composition.

The aforementioned light-curing composition is preferred because the polymer particles referred to above, when used in light-curing compositions, have proven to be particularly advantageous in reducing the surface gloss of dental components produced therefrom. It has proven to be particularly advantageous if the polymer particles consist of polymers of monomers which themselves do not form part of the liquid monomer composition, or which have a mass fraction of less than 10% in the liquid monomer composition and, as a result, have comparatively little influence on the physicochemical properties of the polymerized monomer composition. Without wishing to be bound by this theory, it is presumed that in the case of correspondingly preferred light-curing compositions, the gradient of the chemical composition between the solid polymer particles and the material of the dental component obtained by polymerization from the liquid monomer composition is particularly pronounced, wherein the optical properties in the small domains introduced by the solid polymer particles are particularly different from those obtained when the liquid monomer composition is cured.

For this reason, light-curing compositions are also preferred, wherein the solid polymer particles are at least partially transparent polymer particles whose refractive index differs from the refractive index obtained when the liquid monomer composition is polymerized.

Preference is given to a light-curing composition according to the invention, wherein the solid polymer particles have a substantially spherical particle shape and have been preferably produced by spray-drying.

Corresponding light-curing compositions are regarded as preferred because a corresponding spherical, i.e. ball-like, particle shape has regularly been found when examining the polymer particles used in the experiments. Particularly in light of the presumed effect of the solid polymer particles in the cured light-curing composition, it is assumed that the relatively uniform spatial expansion in the spherical polymer particles contributes to the altered scattering property of the surfaces, for example in comparison with flat, disk-shaped polymer particles.

Preference is given to a light-curing composition according to the invention, wherein the radically polymerizable monomers are selected from the group consisting of monofunctional (meth)acrylates and polyfunctional (meth)acrylates, preferably selected from the group consisting of diurethane dimethacrylate, tris(2-acryloyloxyethyl) isocyanurate, alkoxylated bisphenol A dimethacrylate, tricyclo[5.2.1.0²⁶]decanedimethanol diacrylate and dicyclopentanylmethyl acrylates,
and/or
wherein the radically polymerizable monomers do not contain any silicon atoms.

The corresponding light-curing compositions are preferred because particularly good results have been achieved in practice with the corresponding radically polymerizable monomers. These radically polymerizable monomers are also established components in the field of dental chemistry, for which comprehensive approvals are available and the hazard potential of which is well researched. Moreover, these (meth)acrylates can be photopolymerized very efficiently and, due to the wide range of possible components, allow particularly flexible adjustment of the physicochemical properties. Very particular preference is given to a light-curing composition which contains diurethane dimethacrylate, tris(2-acryloyloxyethyl)isocyanurate, alkoxylated bisphenol A dimethacrylate, tricyclo[5.2.1.0²⁶]decanedimethanol diacrylate and dicyclopentanylmethyl acrylates, since they have been identified in separate experiments as suitable starting materials for the production of dental components which have particularly favorable mechanical properties.

Preference is given to a light-curing composition according to the invention wherein the liquid monomer composition comprises radically polymerizable monomers whose boiling point at 101.3 kPa pressure is above 100° C., preferably above 120° C., particularly preferably above 140° C.

Corresponding light-curing compositions are very particularly preferred because the inventors have recognized that the problem of surface gloss in dental components occurs primarily when radically polymerizable monomers are used which have a low vapor pressure, i.e. their boiling point at ambient pressure is above a certain temperature. At the same time, however, high-boiling monomers are regularly essential for adjusting the physicochemical properties of dental components. Although undesirable gloss effects are also seen when other radically polymerizable monomers are used, the reduction in gloss achievable with the light-curing compositions according to the invention is particularly pronounced for the monomers defined here.

The liquid monomer composition preferably comprises a mass fraction of at least 20%, preferably at least 40%, very particularly preferably at least 60%, of the radically polymerizable monomers having the boiling point defined above. Without wishing to be bound by this theory, the inventors assume that the increased gloss tendency of high-boiling monomers is related to the process temperature which, for example, arises occurs locally in the SLA method at the moment of light-induced polymerization, for example. The increased gloss tendency appears to be more pronounced the further the boiling point of the radically polymerizable monomers is from the temperature of about 70 to 80° C. occurring during polymerization.

A light-curing composition according to the invention is preferred wherein the mass fraction of the liquid monomer composition is 70% or more, preferably 80% or more, particularly preferably 85% or more,
and/or
wherein the combined mass fraction of the photoinitiators is in the range from 0.01% to 5%, preferably 0.1% to 2%, particularly preferably 0.2% to 1%,
and/or
wherein the combined mass fraction of the solid polymer particles is in the range from 0.2% to 20%, preferably 0.5% to 15%, particularly preferably 1% to 10%.

Corresponding light-curing compositions are preferred because light-curing compositions with a high mass fraction of the liquid monomer composition exhibit particularly good processability and at the same time a relatively low proportion of solid polymer particles and can also be produced particularly easily and cost-effectively. In addition, it is particularly cost-efficient to use the smallest possible amounts of photoinitiators, which is also very conducive to the material obtained being stable in storage. Usually, in the case of light-curing compositions according to the invention, satisfactory results are advantageously achieved even with small proportions of photoinitiators, and the inventors have observed that even small amounts of solid polymer particles which are added to the light-curing composition have a particularly pronounced effect on gloss properties even in the case of monomer-rich compositions.

Preference is given to a light-curing composition according to the invention wherein the solid polymer particles have a particle diameter in the range from 0.5 to 2.5 pm, preferably in the range from 0.7 to 2 μm,
and/or
wherein the solid polymer particles have a d50 value in the range from 0.7 to 2 μm, preferably in the range from 0.8 to 1.6 μm, particularly preferably in the range from 0.9 to 1.2 μm.

By choosing suitable particle sizes for the solid polymer particles, the rheological properties of the light-curing composition in the liquid state, in particular the dynamic viscosity at 23° C., can advantageously be adjusted in a targeted manner. For the same mass fraction of solid polymer particles, smaller particle diameters also regularly correlate with a significantly more homogeneous distribution in the material. In this respect, the inventors have found that a particularly pronounced reduction in gloss value can be achieved with particularly small particles, which is attributed to this particularly homogeneous distribution in the light-curing composition.

A light-curing composition according to the invention is preferred, wherein the light-curing composition has at 23° C. a dynamic viscosity in the range from 0.1 to 10 Pa s, preferably in the range from 0.5 to 5 Pa s, particularly preferably in the range from 0.7 to 2.5 Pa s.

The light-curing composition specified above is preferred because it can be processed particularly efficiently, and at the same time enables sufficient stabilization of the solid polymer particles in the light-curing composition. By preventing premature sedimentation of the solid polymer particles, the viscosity defined above contributes to an increased storage stability, which is particularly advantageous in the light-curing compositions according to the invention. In the context of the present invention, the dynamic viscosity at 23° C. is determined using an Anton Paar rheometer (Physica NCR 301) according to DIN 1342-2; 2003-11 Newtonian liquids and DIN 1342-3; 2003-11 Non-Newtonian liquids.

Preference is given to a light-curing composition according to the invention wherein the photoinitiators can initiate the polymerization reaction upon irradiation with electromagnetic radiation in the wavelength range from 200 to 500 nm, preferably in the wavelength range from 350 to 450 nm, particularly preferably in the wavelength range from 380 to 420 nm.

Corresponding photoinitiators regularly have a particularly high compatibility with the (meth)acrylates commonly used. The use of radiation in the wavelength range between ultraviolet and blue is also particularly preferred because this radiation is more energetic than, for example, red radiation and thus regularly enables cleaner and faster initiation of photoinduced curing.

Preference is given to a light-curing composition according to the invention additionally comprising one or more additives with a combined mass fraction in the range from 0.01 to 10%, preferably in the range from 0.1 to 5%, particularly preferably 0.2 to 2%, wherein the additives are selected from the group consisting of fillers, dyes, pigments, flow promoters, thixotropic agents, thickeners and stabilizers.

A particular advantage of the light-curing composition according to the invention is that further additives can be added without adversely affecting the positive effects on the gloss properties of the dental component to be produced.

The invention further relates to a method for producing a light-curing composition for the production of dental components in a DLP method or SLA method, preferably a light-curing composition according to the invention, comprising the steps of:
A) producing or providing a liquid monomer composition comprising one or more radically polymerizable monomers,
B) producing or providing solid polymer agglomerates comprising agglomerated solid polymer particles with a particle diameter in the range from 0.4 to 4 μm,
C) mixing the components produced or provided in steps A and B to obtain a basic mixture comprising on the basis of the total mass of the basic mixture: the liquid monomer composition with a mass fraction of 60% or more, and the solid polymer agglomerates with a combined mass fraction of 0.1 to 30%,
D) mechanically treating the basic mixture to break down the solid polymer agglomerates and to disperse the polymer particles in the liquid monomer composition.

According to the invention, the liquid monomer composition in step A) and the solid polymer agglomerates in step B) can be produced during the course of the method or provided separately, for example by purchasing them from a supplier. It is essential for the method according to the invention that the solid polymer particles are not used—i.e. introduced into the liquid monomer composition—in isolated form but in the form of solid polymer agglomerates, which are sometimes also referred to as agglomerated polymer beads. These solid polymer agglomerates comprise a plurality of solid polymer particles as primary particles and are mixed with the monomer composition in step C) in order to obtain a basic mixture.

In this basic mixture, the solid polymer agglomerates tend to be not homogeneously dispersed in the basic mixture, but instead float in the liquid monomer composition. In this state, the basic mixture is not suitable for use in an SLA method or DLP method and the dental component obtained does not show any improvements with regard to the gloss properties. Only by mechanically treating the basic mixture in step D) is a light-curing composition obtained that exhibits the positive effect according to the invention.

The basic mixture is mechanically treated to break down the solid polymer agglomerates and disperse the polymer particles in the liquid monomer composition. The mechanical treatment disintegrates the polymer agglomerates into the primary particles, i.e. the solid polymer particles, and a substantially homogeneous dispersion of the solid polymer particles in light-curing compositions is achieved.

In the method according to the invention, the photoinitiators required for light curing still have to be added to the basic mixture in order to produce the light-curing composition. The time at which the photoinitiators are mixed in is not relevant. This can be done, for example, after the mechanical treatment or even before the two components are combined, for example by adding said photoinitiators to the liquid monomer mixture, although in all cases it is expedient to ensure thorough mixing.

The method according to the invention makes it possible to produce light-curing compositions according to the invention in a cost-effective and reproducible manner. In this respect, the method can be carried out using comparatively common devices, and the required starting materials can be handled and stored comparatively easily, since the operating personnel do not come into contact with small nanoparticles in dry or dusty form when carrying out the method, thus ensuring a high level of operational safety.

Preference is given to a method according to the invention wherein the polymer agglomerates have a particle diameter in the range from 5 to 200 μm, preferably in the range from 10 to 150 μm, particularly preferably in the range from 15 to 75 μm.

If sufficiently small polymer agglomerates are used, they can be better and more uniformly distributed in the basic mixture even before mechanical treatment and are easier to break down by mechanical treatment. As a result, more homogeneous light-curing compositions are advantageously obtained. However, it should be noted that larger polymer agglomerates can certainly have advantages, particularly in terms of ease of handling and with regard to health aspects, especially if dust formation occurs during processing.

Preference is given to a method according to the invention wherein the polymer agglomerates have a substantially spherical shape.

Preference is given to a method according to the invention wherein the mechanical treatment is carried out by a milling process and/or stirring process and/or rolling process, in particular with a ball mill and/or a dissolver and/or a three-roll mill, preferably in a rolling process, in particular in a three-roll mill.

The basic mixture can in principle be mechanically treated by all methods suitable for breaking down the solid polymer agglomerates. In practice, grinding processes, stirring processes and rolling processes have proven to be particularly useful for this purpose, wherein the use of a three-roll process, that is to say using a three-roll mill, is particularly preferred, since this enables particularly efficient and targeted destruction of the agglomerates after they have been incorporated into the basic mixture by setting a gap between the rolls that is as narrow as possible. In particular, the use of a three-roll mill permits sufficient destruction of the polymer agglomerates, which in some cases is more difficult to achieve over the entire volume of the basic mixture, especially in stirring processes.

The person skilled in the art will adjust the intensity of the mechanical treatment based on routine experiments in order to identify the optimum load levels depending on the materials used. If a sufficiently large mechanical load is not selected, significant proportions of polymer agglomerates may remain, thereby reducing the technical effect of the light-curing composition according to the invention. Furthermore, it has been observed that an excessive mechanical load on the basic mixture can also lead to deterioration in the overall properties of the light-curing composition produced and a decrease in the quality of the resulting dental component.

A light-curing composition produced according to the above method is particularly preferred.

The invention also relates to the use of a light-curing composition according to the invention for producing dental components with matte surfaces in a DLP method or SLA method, wherein the surface of the dental component has a gloss value of 10 GU or less, preferably 5 GU or less, particularly preferably 2 GU or less.

Surprisingly, it has been found that by using a light-curing composition according to the invention in a DLP method or SLA method, dental components can be obtained which have particularly favorable gloss properties and a matte surface. In the context of the present invention, gloss properties are indicated in GU, i.e. gloss unit, with which the person skilled in the art is familiar.

In the context of the present invention, gloss values are determined by a gloss measurement method using a device from BYK of the micro-TRI gloss type with a 60° angle.

Also disclosed is the use of solid polymer agglomerates comprising agglomerated solid polymer particles with a particle diameter in the range from 0.4 to 4 μm in the production of light-curing compositions for the production of dental components in a DLP method or SLA method, preferably for producing light-curing compositions according to the invention.

Surprisingly, it has been found that the use of solid polymer agglomerates in the production of light-curing compositions makes it possible to obtain light-curing compositions according to the invention particularly efficiently without undesirable dust formation occurring during handling of the solid polymer particles.

Also disclosed is the use of solid polymer particles with a particle diameter in the range from 0.4 to 4 μm in a light-curing composition for the production of dental components in a DLP method or SLA method for reducing the gloss properties of the dental components obtained by light curing.

Further disclosed is an uncoated dental component produced in a DLP method or SLA method from a composition according to the invention, wherein the surface of the dental component has a gloss value of 10 GU or less, preferably 5 GU or less, particularly preferably 2 GU or less, and wherein the surface has an average surface roughness Ra of 2.5 μm or less, preferably 2.0 μm or less. Ra is determined here in accordance with the specifications of DIN EN ISO 4287:2010.

These uncoated dental components, which have been produced from the light-curing composition according to the invention in a DLP method or SLA method, have particularly advantageous gloss properties on their surface and do not exhibit any increased surface roughness, which is evidenced by an average surface roughness level Ra of 2.5 μm or less. This advantageously generates particularly smooth surfaces on which, for example, molding composition can be applied and later removed again without leaving any residue. Advantageously, corresponding uncoated dental components therefore also have a large contact angle with water, which means that they can be cleaned particularly efficiently.

Additionally disclosed is a system for producing a dental component in a DLP method or SLA method, comprising a DLP printer or an SLA printer with a liquid reservoir for receiving a light-curing composition, wherein the liquid reservoir comprises the light-curing composition according to the invention.

Finally disclosed is a kit for producing a light-curing composition according to the invention, comprising as separate components in separate containers:
U solid polymer agglomerates comprising agglomerated solid polymer particles with a particle diameter in the range from 0.4 to 4 μm, and
V a liquid monomer composition comprising one or more radically polymerizable monomers.

As explained above, particularly good results with light-curing compositions according to the invention are achieved when the solid polymer particles are dispersed as uniformly as possible in the light-curing composition. Especially when the dynamic viscosities of the light-curing composition are low, this property may deteriorate over prolonged periods of continued storage as the solid polymer particles slowly settle in the liquid monomer composition. Accordingly, it is particularly advantageous to provide the components for the light-curing composition according to the invention to the end user, for example the dentist or the laboratory technician, as separate components from which a light-curing composition according to the invention can be produced by means of the method according to the invention. Advantageously, the solid polymer agglomerates are large enough to enable comparatively safe handling in the laboratory. The dentist or dental technician only has to mix together the two components and ensure by mechanical treatment that the solid polymer agglomerates are broken down into the solid polymer particles, which are then dispersed in the light-curing composition.

Optionally, the kit can additionally comprise, as a further component W, one or more photoinitiators, wherein these can alternatively also be added to the liquid monomer composition. The advantage of separate provision, which requires subsequent mixing of the components U, V and W, is a further increase in storage stability, since premature polymerization of the monomer composition cannot occur as a result of undesired irradiation.

In the drawings:

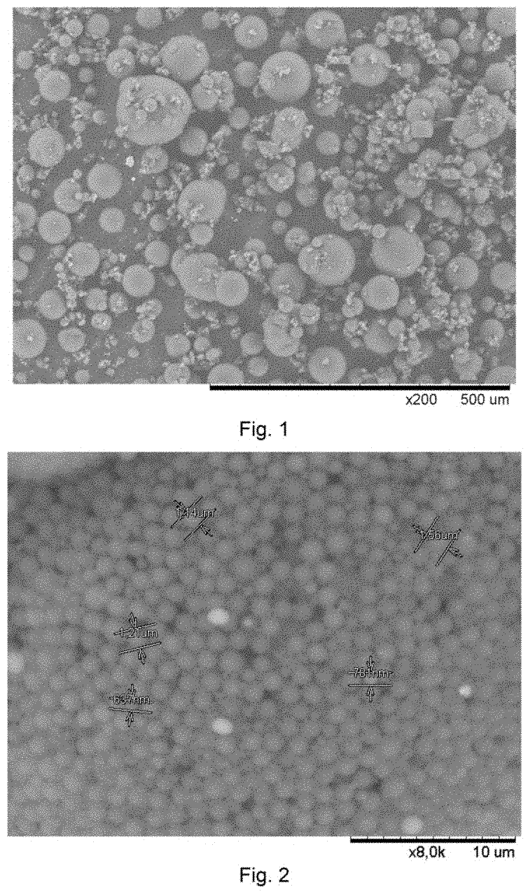

FIG. 1 shows a micrograph of spray-dried polymer agglomerates of polymethyl methacrylate at 200×magnification;

FIG. 2 shows a micrograph of the primary particles of a spray-dried polymer agglomerate of polymethyl methacrylate at 8000×magnification;

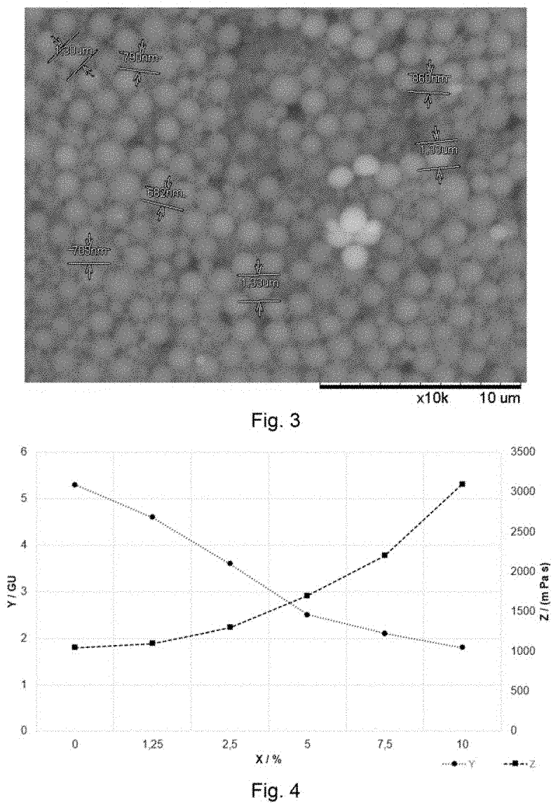

FIG. 3 shows a micrograph of the primary particles of a spray-dried polymer agglomerate of polymethyl methacrylate at 10000×magnification;

FIG. 4 shows a plot of the experimentally determined gloss number (Y) of test specimens produced from light-curing compositions, as well as the dynamic viscosity (Z) of the corresponding light-curing compositions plotted against the mass fraction of solid polymer particles (X) in the light-curing com positions.

The invention and preferred embodiments of the invention are explained and described in more detail below with reference to experiments.

To demonstrate the surprising technical effect of the light-curing composition according to the invention, a reference system is considered below which is regularly used by the inventors in order to investigate the effects of additives, for example. Here, the reference system comprises in particular five radically polymerizable monomers which have very different chemical structures and which represent a broad spectrum of (meth)acrylates used particularly frequently in practice. For the solid polymer particles, PMMA particles were selected in the reference system because they were available as sufficiently small particles and solid PMMA is, from experience, relatively representative of polymerized (meth)acrylates. Neither in the experiments shown here nor in the accompanying experiments carried out by the inventors were any observations made that would have called into question the assumption that the results are transferable to other systems.

The reference system uses the following:
a liquid monomer composition consisting of a mixture of a first and a second monomer mixture in a mixing ratio of 1 to 1.67 by mass, wherein the first monomer mixture consists of equal mass fractions of tris(2-acryloyloxyethyl)isocyanurate, dicyclopentanyl methylacrylate and tricyclo[5.2.1.0²⁶]decanedimethanol diacrylate, and wherein the second monomer mixture consists of bisphenol A ethoxylate dimethacrylate and diurethane dimethacrylate in a mass ratio of 2 to 1.
phenylbis(2,4,6-trimethylbenzoyl)phosphine oxide as photoinitiator;
an additive composition consisting of 2-hydroxy-4-methoxybenzophenone (additive 1); titanium (IV) oxide (additive 2), flame black (additive 3) and 5% PV true blue A2R in diurethane dimethacrylate (additive 4); and
polymer particles of polymethyl methacrylate, i.e. the polymer of methyl methacrylate.

From these substances, light-curing compositions were produced as listed in Table 1, wherein the values are given as a mass fraction in percent. Of these compositions, Ex. 1 to Ex. 5 are light-curing compositions according to the invention, whereas the composition Comp. 1 is a comparative composition which contains no solid polymer particles.

TABLE 1
Composition of the investigated light-curing compositions
with different contents of solid polymer particles.

| | Comp. | Ex. | Ex. | Ex. | Ex. | Ex. |
|---|---|---|---|---|---|---|
| Substance | 1 | 1 | 2 | 3 | 4 | 5 |
| Monomer composition | 98.22 | 97.00 | 95.76 | 93.44 | 91.21 | 88.26 |
| Photoinitiator | 0.82 | 0.80 | 0.80 | 0.78 | 0.76 | 0.80 |
| Additive 1 | 0.35 | 0.34 | 0.34 | 0.33 | 0.32 | 0.34 |
| Additive 2 | 0.55 | 0.54 | 0.54 | 0.53 | 0.51 | 0.54 |
| Additive 3 | 0.03 | 0.03 | 0.03 | 0.03 | 0.03 | 0.03 |
| Additive 4 | 0.03 | 0.03 | 0.03 | 0.03 | 0.03 | 0.03 |
| Polymer particles | 0.00 | 1.25 | 2.49 | 4.87 | 7.13 | 10.00 |
| Total | 100 | 99.99 | 99.99 | 100.01 | 99.99 | 100 |

Minimal deviation in the sum of all components from 100% is due to rounding, but, as understood by the person skilled in the art, does not affect the validity of the experiments, especially since it would not make sense from the point of view of a person skilled in the art to specify the mass fractions more precisely than the production method allows.

The liquid monomer composition comprises five radically polymerizable monomers, and is produced by mixing the components. The photoinitiator is suitable for causing polymerization of the liquid monomer composition by irradiating the composition with light. The polymer particles are solid, spherical polymer particles with a diameter in the range from 0.7 to 2.0 μm. The compositions described above were produced by the method according to the invention, wherein the polymer particles were introduced into the compositions by adding spherical polymer agglomerates with a diameter of about 15 to 75 μm. The additives and the photoinitiator were added to the liquid monomer composition before being mixed with the polymer agglomerates. Mechanical treatment of the basic mixture to break down the solid polymer agglomerates was carried out using a three-roll process wherein the mixture was homogenized twice through a roll gap of 5 μm. As a result of the mechanical treatment, the polymer particles were present in the compositions according to the invention in dispersed form as non-agglomerated particles, wherein no concentration gradients of solid polymer particles were observed over the volume of the light-curing compositions.

FIGS. 1 to 3 show micrographs of the polymer agglomerates used at various magnifications (200×, 8000× and 10000×), wherein the solid polymer particles can be clearly seen as primary particles of the polymer agglomerates.

For the samples produced, the dynamic viscosity (Z) at 23° C. was determined using a Rheometer (Anton Paar, Physica NCR 301, viscosity ranges 200-3000 m·Pa s at 100/s, 23° C.) according to DIN 1342-2; 2003-11 Newtonian liquids and DIN 1342-3; 2003-11 Non-Newtonian liquids. The results obtained are collated in Table 2.

TABLE 2
Dynamic viscosities (Z) of the investigated light-curing compositions
with different contents of solid polymer particles.

| | Dynamic |
|---|---|
| | viscosity |
| Sample | (Z)/(m Pa s) |
| Comp. 1 | 1050 |
| Ex. 1 | 1100 |
| Ex. 2 | 1300 |
| Ex. 3 | 1700 |
| Ex. 4 | 2200 |
| Ex. 5 | 3100 |

The measured values clearly indicate that the viscosity of the light-curing compositions according to the invention is still sufficiently low, even for a mass fraction of the solid polymer particles of 10%, for use in a SLA or DLP method. By extrapolating the viscosity increase as a function of the mass fraction of the polymer particles, it can be estimated that mass fractions of up to 30% are feasible without losing the fundamental suitability of the composition according to the invention for use in SLA or DLP methods.

At the same time, it has proven advantageous that the viscosity of the light-curing compositions can be adjusted particularly easily by adding the solid polymer particles in light-curing compositions according to the invention and can be adapted to the respective requirements without needing, for example, inorganic additives.

To investigate the gloss property of the dental components produced from the light-curing compositions according to the invention, test specimens measuring 60 mm×10 mm×3.3 mm were produced by a DLP method using a DLP printer from the Kulzer company of the cara Print 4.0 type. The test specimen was manufactured in each case horizontally or vertically, i.e. the material layers generated in the DLP method were built up parallel or perpendicular to the surface measured as part of the gloss measurement.

The gloss figures were determined using a gloss measurement method which was performed with a device from the BYK company of the micro-TRI gloss type using a 60° angle. The values obtained are summarized in Table 3.

TABLE 3
Gloss number (Y) of the investigated test specimens produced
from light-curing compositions with different contents of
solid polymer particles, including standard deviation.

| | Gloss number | Gloss number |
|---|---|---|
| | (Y)/GU | (Y)/GU |
| | (horizontally | (vertically |
| Sample | manufactured) | manufactured) |
| Comp. 1 | 5.3 ± 1.2 | 43.4 ± 3.2 |
| Ex. 1 | 4.6 ± 1.3 | 9.5± 1.1 |
| Ex. 2 | 3.6 ± 0.8 | 2.6 ± 0.7 |
| Ex. 3 | 2.5 ± 0.6 | — |
| Ex. 4 | 2.1 ± 0.6 | — |
| Ex. 5 | 1.8 ± 0.7 | 1.2 ± 0.9 |

The values of the gloss number (Y) tabulated above for horizontal manufacturing are plotted in FIG. 4 together with the dynamic viscosities (Z) against the mass fraction of the solid polymer particles in the corresponding light-curing compositions (X).

It is clear from the experimental data that even adding small amounts of solid polymer particles with a particle diameter in the range from 0.4 to 4 μm results in an advantageous reduction in the gloss property of the test specimens. The observed effect is particularly pronounced in the case of vertically manufactured test specimens, i.e. when the surface gloss is measured on a surface that is parallel to the built-up layers. Even when a 1.25% mass fraction of solid polymer particles is added, a reduction of more than 75% in the gloss property is observed. The data obtained indicate that this is an effect whose magnitude varies with the amount of solid polymer particles, which means that there is no indication of a possible limit value above which the effect would be observed for the first time. By selecting suitable amounts of solid polymer particles, particularly matte and therefore readily scannable surfaces can be obtained accordingly in the dental components produced from light-curing compositions according to the invention.

In addition, the average surface roughness Ra of selected test specimens was determined. The values obtained are summarized in Table 4.

TABLE 4
Average surface roughness (Ra) of the investigated test
specimens produced from light-curing compositions with
different contents of solid polymer particles.

| | Average |
|---|---|
| | surface |
| | roughness |
| Sample | (Ra)/μm |
| Comp. 1 | 1.36 |
| Ex. 1 | 1.94 |
| Ex. 2 | 1.68 |
| Ex. 3 | 1.75 |
| Ex. 4 | 0.79 |
| Ex. 5 | 1.13 |

It is evident that the average surface roughness of the test specimens produced with the light-curing compositions according to the invention is advantageously not significantly increased compared to the comparative sample. Accordingly, it is advantageously possible to manufacture dental components which have smooth surfaces. Such dental components are particularly easy to clean and make it possible to remove applied molding compositions so as to leave especially little residue.

1. A light-curing composition for producing dental components in a digital light processing (DLP) method or a stereolithography (SLA) method, comprising, based on the total mass of the light-curing composition:
a liquid monomer composition with a mass fraction of 60% or more, comprising one or more radically polymerizable monomers,
one or more photoinitiators with a combined mass fraction in the range from 0.001 to 10%, and
solid polymer particles with a particle diameter in the range from 0.4 to 4 μm and a combined mass fraction in the range from 0.1 to 30%, wherein the solid polymer particles are dispersed in the liquid monomer composition.

2. The light-curing composition according to claim 1, wherein the solid polymer particles comprise polymers of monomers selected from the group consisting of monofunctional (meth)acrylates, polyfunctional (meth)acrylates, and polymethyl methacrylate, wherein the solid polymer particles comprise polymers of monomers that are not part of the liquid monomer composition.

3. The light-curing composition according to claim 1, wherein the solid polymer particles are present in the light-curing composition as non-agglomerated particles, wherein the solid polymer particles are dispersed substantially homogeneously in the liquid monomer composition.

4. The light-curing composition according to claim 1, wherein the liquid monomer composition comprises radically polymerizable monomers whose boiling point at 101.3 kPa pressure is one or more above 100° C., above 120° C., or above 140° C.

5. The light-curing composition according to claim 1, wherein the solid polymer particles have a particle diameter in the range from 0.5 to 2.5 μm, or in the range from 0.7 to 2 μm.

6. The light-curing composition according to claim 1, wherein the light-curing composition has at 23° C. a dynamic viscosity in the range from 0.1 to 10 Pa s, in the range from 0.5 to 5 Pa s, or in the range from 0.7 to 2.5 Pa s.

7. The light-curing composition according to claim 1, wherein the solid polymer particles have a substantially spherical particle shape and have been produced by spray-drying.

8. A method for producing a light-curing composition for the production of dental components in a digital light processing (DLP) method or a stereolithography (SLA) method, comprising the steps of:
A) producing or providing a liquid monomer composition comprising one or more radically polymerizable monomers,
B) producing or providing solid polymer agglomerates comprising agglomerated solid polymer particles with a particle diameter in the range from 0.4 to 4 μm,
C) mixing the components produced or provided in steps A and B in order to obtain a basic mixture, comprising on the basis of the total mass of the basic mixture:
the liquid monomer composition with a mass fraction of 60% or more, and
the solid polymer agglomerates with a combined mass fraction of 0.1 to 30%,
E) mechanically treating the basic mixture to break down the solid polymer agglomerates and to disperse the polymer particles in the liquid monomer composition.

9. The method according to claim 8, wherein the mechanical treatment is carried out by a milling process and/or stirring process and/or rolling process, in particular with a ball mill and/or a dissolver and/or a three-roll mill, preferably in a rolling process, in particular in a three-roll mill.

10. Use of a light-curing composition according to claim 1 for producing dental components with matte surfaces in a digital light processing (DLP) method or as stereolithography (SLA) method, wherein the surface of the dental component has a gloss value of 10 GU or less, 5 GU or less, or 2 GU or less.

11. Use of solid polymer particles with a particle diameter in the range from 0.4 to 4 μm in a light-curing composition for the production of dental components in a digital light processing (DLP) method or a stereolithography (SLA) method for reducing the gloss of the dental components obtained by light curing.

12. A kit for producing a light-curing composition according to claim 1, comprising as separate components in separate containers:
solid polymer agglomerates comprising agglomerated solid polymer particles with a particle diameter in the range from 0.4 to 4 μm, and
a liquid monomer composition comprising one or more radically polymerizable monomers.
